(19) United States
(12) Patent Application Publication
(10) Pub. No.: US 2009/0169532 A1
(43) Pub. Date: Jul. 2, 2009

(54) CURABLE BONE CEMENT

(76) Inventors: Jackie Y. Ying, Nanos (SG); Shona Pek, Nanos (SG); Motoichi Kurisawa, Nanos (SG); Joo Eun Chung, Nanos (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/280,777

(22) PCT Filed: Feb. 27, 2006
(86) PCT No.: PCT/SG2006/000039

§ 371 Date: Jan. 14, 2009

(51) Int. Cl. A61K 35/14; A61K 47/42; A61K 47/36; A61K 47/34

(52) U.S. Cl. 424/93.72; 514/773; 514/777; 523/116

(57) ABSTRACT

The present invention describes a curable bone cement. The cement comprises a curable polymeric binder and a filler, and is capable of curing without substantial evolution of heat on exposure to a curing agent. The binder comprises phenol groups which are capable of reacting in order to cure the cement.

### TECHNICAL FIELD

The present invention relates to a curable composition for use in bone cement applications.

### BACKGROUND OF THE INVENTION

Many clinical procedures such as maxillofacial surgery and osteochondral surgery require the use of bone cements to fill bone defects and deficiencies. Otherwise, the bone defects and deficiencies would not heal properly, preventing the return of normal function. Various synthetic bone substitutes have been developed for this purpose, some of which have been produced in an injectable form, so as to enable minimally invasive surgery. The main use of injectable bone substitutes include spinal fusion, bone and joint defects, osteoporotic fractures, revision surgery and vertebroplasty. A common disadvantage of injectable bone substitutes is that they generate heat during the process of curing. This heat has the potential to damage surrounding tissue.

There is therefore a need for a curable bone substitute that does not generate heat when curing

### OBJECT OF THE INVENTION

It is the object of the present invention to overcome or substantially ameliorate at least one of the above disadvantages.

### SUMMARY OF THE INVENTION

Disclosed herein is a curable bone cement comprising a curable binder and a filler, wherein the cement (and/or the binder) is capable of curing without substantial evolution of heat. The cement may be capable of curing on exposure to (e.g. combination with, mixing with or addition of) a curing agent. The curing agent may be a reagent or may be a catalyst. The binder and the filler may be biocompatible. The curing agent may be biocompatible.

The binder may be crosslinkable without substantial evolution of heat. It may be a polymeric or oligomeric binder. It may be crosslinkable by means of an oxidant, e.g. a mild oxidant. It may comprise —C₆R′₄OR groups (i.e. phenol groups), wherein R and each R′ may independently be hydrogen, an alkyl group, an aryl group or an acyl group, and R′ may also be OH. Each R′ may be the same as or different to each other R′, provided that at least one R′, for example an R′ ortho to the OR group, is hydrogen. R and R′ may be such that one —C₆R′₄OR group is capable of oxidatively coupling with another —C₆R′₄OR group. The —C₆R′₄OR groups may be for example —C₆H₄OH groups. The binder may comprise a combination, a complex, a reaction product or a conjugate, of a polymeric species and a phenolic species. The phenolic species may be a polyphenol. Suitable phenolic species include tyramine, catechin, epicatechin, gallic acid and epigallocatechin gallate (EGCG), as well as mixtures of any two or more thereof. The polymeric species may be a biopolymer or a derivative thereof. It may be for example hyaluronic acid, a polyamine or a polypeptide, such as gelatin and/or collagen. The filler may be an apatite filler, for example hydroxyapatite, carbonated apatite, fluoroapatite, or any form of modified apatite or a combination of several types of apatite in any proportion, or may be some other mineral filler for example silica, alumina, zirconia, calcium phosphate, talc, calcium carbonate, mica.

In a first aspect of the invention there is provided a curable bone cement comprising a curable polymeric binder and a filler, wherein the cement is capable of curing without substantial evolution of heat on exposure to a curing agent, said binder comprising phenol groups which are capable of reacting in order to cure the cement. The phenol groups may be capable of oxidatively coupling in order to cure the polymeric binder. The phenol groups may be —C₆R′₄OR groups, wherein R and each R′ are independently hydrogen, an alkyl group, an aryl group or an acyl group, and R′ may also be OH, and each R′ is the same as or different to each other R′, provided that at least one R′, for example an R′ ortho to the OR group, is hydrogen, and wherein R and R′ are such that one —C₆R′₄OR group is capable of oxidatively coupling with another —C₆R′₄OR group.

At least some of the —C₆R′₄OR groups may be —C₆H₄OH groups. The binder may comprise for example a hyaluronic acid-tyramine (HA-Tyr) conjugate, a gelatin-Tyr conjugate or a hyaluronic acid-epigallocatechin gallate (HA-EGCG) conjugate. The filler may comprise a mineral filler, for example silica, alumina, zirconia, talc, an apatite or a mixture of any two or more of these. The filler may additionally or alternatively comprise particles capable of releasing a drug, a protein and/or a growth factor. The particles may be controlled release particles. Such particles may be useful for enhancing healing of the bone or of tissue surrounding the bone. Examples of suitable apatite fillers include hydroxyapatite, carbonated apatite, fluoroapatite, or any form of modified apatite or a combination of two or more types of apatite in any proportion. An example of a suitable apatite filler is a mixture of hydroxyapatite (HAP) and carbonated apatite (CAP). The curing agent may be selected so that the bone cement is capable of curing in acceptable time at the temperature of use (e.g. at the body temperature into which the bone cement is injected). The bone cement may be capable of curing in between about 10 seconds and about 30 minutes, or between about 20 seconds and 1 minute on exposure to the curing agent at the body temperature of a patient in which the cement is cured. The curing agent may comprise an oxidant. The curing agent may be an agent for oxidative coupling of phenolic groups. The curing agent may be a mild oxidant so that curing of the cement may be accomplished without substantial evolution of heat. The curing agent may comprise an enzyme, e.g. a peroxidase. It may comprise a peroxide. It may comprise a combination of a peroxide and an enzyme e.g. a peroxidase such as horse radish peroxidase (HRP). For example, the curing agent may comprise hydrogen peroxide and horse radish peroxidase. Other suitable curing agents comprise glutathione peroxidase, myeloperoxidase, tyrosinase or laccase in combination with or without a peroxide.

The bone cement may additionally comprise one or more further component such as collagen, a silicate, a protein (e.g. growth factor) and platelets.

The bone cement may be injectable. It may be in the form of a paste, or a slurry or some other viscous preparation. It may show shear thinning (pseudoplastic) rheology. It may show plastic rheology i.e. it may exhibit a finite yield stress. Once mixed with the curing agent, the bone cement may be injectable. It may be in the form of a paste, or a slurry or some other viscous preparation.

In an embodiment the curable bone cement comprises:
a conjugate of hyaluronic acid with a compound selected from the group consisting of tyramine, catechin, epicatechin, gallic acid and epigallocatechin gallate, and mixtures of any two or more thereof, and
an apatite filler,
   whereby the cement is curable on exposure to a peroxide and a peroxidase enzyme without substantial evolution of heat.

In another embodiment, the curable bone cement comprises a hyaluronic acid-tyramine (HA-Tyr) conjugate and an apatite filler, whereby the cement is curable on exposure to hydrogen peroxide and horse radish peroxidase without substantial evolution of heat.

In another embodiment the curable bone cement comprises:
a conjugate of a polyamine or a polypeptide such as gelatin and/or collagen with a compound selected from the group consisting of tyramine, catechin, epicatechin, gallic acid and epigallocatechin gallate, and mixtures of any two or more thereof, and
an apatite filler,
   whereby the cement is curable on exposure to a peroxide and a peroxidase enzyme without substantial evolution of heat.

The curable bone cement may contain a mixture of gelatin-Tyr, HA-Tyr and/or an apatite filler.

There is also provided the use of a curable binder and a filler for the manufacture of a bone cement for use in repairing bones, said binder comprising phenol groups with at least one hydrogen atom attached to the aromatic ring thereof.

There is also provided a kit comprising a curable bone cement according to the first aspect and a curing agent, whereby said curing agent is capable of causing the curable bone cement to cure without substantial evolution of heat. The ratio of the bone cement to the curing agent in the kit may be such that, when the bone cement and the curing agent of the kit are combined in said ratio, the bone cement is capable of curing in between about 10 seconds and about 30 minutes at the body temperature of a patient. There is further provided a catalysed bone cement comprising the curable bone cement combined with the curing agent.

In a second aspect of the invention there is provided a process for making a curable bone cement comprising combining a solution of a curable binder with a filler, and optionally with one or more further component such as collagen, a silicate, a protein (e.g. growth factor) and platelets, said binder comprising phenol groups which are capable of reacting in order to cure the cement. The curable binder and the filler may be as described above. Thus for example the filler may comprise an apatite or a mixture of two or more apatites. The step of combining may comprise preparing a solution of the curable binder. It may comprise combining the solution of the curable binder with the filler.

The process may also comprise the step of making the curable binder. This may comprise coupling a phenolic species with a polymeric species. The polymeric species may be a biopolymer, e.g. hyaluronic acid, or a derivative thereof. It may be a polyamine or a polypeptide, e.g. gelatin or collagen. The phenolic species may comprise one or more —C₆R′₄OR groups. It may or may not comprise an amine functional group.

In an embodiment, the process comprises combining a solution of a curable binder, such as a hyaluronic acid-tyramine (HA-Tyr) conjugate, with an apatite filler, and optionally with one or more further component such as collagen, a silicate, a protein (e.g. growth factor) and platelets.

In another embodiment the process comprises:
coupling a phenolic species with a polymeric species to form a curable binder; and
combining a solution of the curable binder with a filler, and optionally with one or more flirter components such as collagen, a silicate, a protein (e.g. growth factor) and platelets, said binder comprising phenol groups which are capable of reacting in order to cure the cement.

The invention also provides a curable bone cement when made by the process of the second aspect.

There is also provided a process for making a catalysed bone cement comprising providing a curable bone cement according to the first aspect and exposing (e.g. combining, mixing or adding) said curable bone cement to a curing agent, whereby said curing agent is capable of causing the curable bone cement to cure without substantial evolution of heat. The step of providing the curable bone cement may comprise preparing said curable bone cement, for example by the process of the second aspect of the invention.

In a third aspect of the invention there is provided a method for curing a bone cement, said bone cement comprising a curable binder and a filler, said binder comprising phenol groups which are capable of reacting in order to cure the cement, said method comprising:
exposing the curable bone cement to a curing agent to form a catalysed bone cement; and
curing the catalysed bone cement without substantial evolution of heat.

The curable binder, the filler and the curing agent may be as described above. Thus for example the filler may comprise an apatite or a mixture of two or more apatites and the curing agent may comprise a peroxide and a peroxidase enzyme. The process may comprise the step of injecting the bone cement into a patient, or otherwise locating the bone cement in and/or on the bone of a patient. This step may be conducted before the step of curing the catalysed bone cement. The curable bone cement and the curing agent may be used in non-toxic amounts in the patient.

The invention also provides a cu bone cement when made by the process of the third aspect of the invention.

In a fourth aspect of the invention there is provided a method for repairing a bone in a patient comprising:
combining a curable bone cement comprising a curable binder and a filler with a curing agent to form a catalysed bone cement, said binder comprising phenol groups which are capable of reacting in order to cure the cement,
injecting said catalysed bone cement onto and/or into said bone; and
curing the catalysed bone cement on and/or in the bone without substantial evolution of heat.

The curable binder, the filler and the curing agent may be as described above. Thus for example the filler may comprise an apatite or a mixture of two or more apatites and the curing agent may comprise a peroxide and a peroxidase enzyme.

### BRIEF DESCRIPTION OF THE DRAWINGS

A preferred form of the present invention will now be described by way of example with reference to the accompanying drawings wherein:

FIG. 1 shows micrographs of bone injected with a bone cement according to the present invention, with staining with (a) H and B, (b) ALP and NFR, and (c) VK and NFR for cement 1 of the example (HA solution plus curing agent) (control) 5 weeks after injection;

FIG. 2 shows micrographs of bone injected with a bone cement according to the present invention, with staining with (a) H and E, (b) ALP and NFR, and (e) VK and NFR for cement 2 of the example (HA solution and apatite powders, plus curing agent) 5 weeks after injection;

FIG. 3 shows micrographs of bone injected with a bone cement according to the present invention, with staining with (a) H and E, (b) ALP and NFR, and (c) VK and NFR for cement 3 of the example (HA solution and apatite powders, and collagen solution, plus curing agent) 5 weeks after injection;

FIG. 4 shows micrographs of bone injected with a bone cement according to the present invention, with staining with (a) H and E, (b) ALP and NFR, and (c) VK and NFR for cement 4 of the example (HA solution, and pre-mixed collagen-apatite solution, plus curing agent) 5 weeks after injection;

FIG. 5 shows a representative crosslinked structure according to the present invention;

FIG. 6 shows a scheme for making a HA-dialkyl acetal conjugate, and

FIG. 7 shows a scheme for making a HA-EGCG conjugate.

### DETAILED DESCRIPTION OF THE PREFERRED EMBODIMENTS

The present invention provides a curable bone cement comprising a curable binder and a filler, wherein the cement (and/or the binder) is capable of curing without substantial evolution of heat. The cement may be capable of curing on exposure to a curing agent. Any or all of the components of the curable binder and of the cueing agent may be pharmaceutically, clinically and/or veterinarily acceptable. They may be non-toxic to a patient in which they are used. They may be biocompatible.

The curable binder may comprise a polymeric species, or macromolecular species, and may also comprise either crosslinking moieties attached to the polymeric species or a crosslinking species mixed with the polymeric species. The polymeric species may be biocompatible. It may be non-toxic. It may for example be a glycosaminoglycan, a polysaccharide, a polycarboxylic acid, chondroitin, chondroitin sulfate, dermatan sulfate, heparan sulfate, heparin, proteoglycans, polyuronic acids (e.g. polypectate, polygalacturonic acid, polyglucuronic acid, pectin, colominic acid, alginate or some other polymeric species, and may be substituted. A suitable polymeric species is hyaluronan or hyaluronic acid, which may be substituted. The substituents may be the crosslinking moieties. The crosslinking moieties way comprise —C₆R′₄OR (i.e. phenol) groups which are capable of reacting in order to cure the cement. In the —C₆R′₄OR groups, R and each R′ may, independently, be hydrogen, an alkyl group, an aryl group or an acyl group, and R′ may also be OH, and each R′ is the same as or different to each other R′, provided that at least one R′, for example ortho to the OR group, is hydrogen, and wherein R and R′ are such that one —C₆R′₄OR group is capable of oxidatively coupling with another —C₆R′₄OR group. The other —C₆R′₄OR group may be attached to a different molecule of the polymeric species, so that the oxidative coupling crosslinks the polymeric species. The alkyl group may be a C1 to C12 or more straight chain alkyl group. It may be a C3 to C12 or more branched or cyclic alkyl group, or may have a mixture of alkyl and cycloalkyl portions (e.g. it may be cyclohexylmethyl). Suitable alkyl groups include methyl, ethyl, propyl etc. It will be understood that other substituents may be used, including alkenyl, alkynyl, aryl, heteroaryl groups etc. The nature of the groups K and R′ should not be such as to prevent oxidative coupling of —C₆R′₄OR groups. Thus for example excessively bulky substitutents, particularly the R′ groups which are on the ring, may inhibit or prevent coupling of the groups due to steric hindrance. Certain R′ groups may inhibit or prevent coupling due to electronic factors. At least some of the —C₆R′₄OR groups may be —C₆H₄OH groups, e.g. p-C₆H₄OH, or —C₆H₂(OH)₃, e.g. 3, 4, 5-trihydroxyphenyl groups. At least some of the phenol groups may be fused ring phenol groups e.g. a chromane structure bearing at least one phenolic OH group.

The binder may be generated by coupling the —C₆R′₄OR groups to a polymeric species (a polymer or an oligomer), optionally a biocompatible or nontoxic polymer or oligomer. The polymeric species may be a biopolymer. It may be a polysaccharide, a polyamine or a polypeptide, e.g. hyaluronic acid, gelatin or collagen. The coupling may comprise reacting the polymeric species with an aminofunctional phenolic species which comprises the C₆R′₄OR group. Thus the amine group may be capable of coupling with a functional group (e.g. carboxylate, haloalkyl etc.) in the polymeric species. A suitable aminofunctional species may have formula H₂N-L-C₆R′₄OR, wherein R and R′ are as described above, and L is a linker group. L may be alkylene, arylene or some other suitable linker group e.g. methylene (—CH₂—), ethylene (—CH₂C₂—), propylene (—CH₂CH₂CH₂—) etc. and may be straight chain, branched or cyclic. A suitable aminofunctional species may be tyramine (Tyr).

Alternatively or additionally, the coupling may comprise reacting the polymeric species with a non-aminofunctional phenolic species, such as a polyphenol. Suitable polyphenols include catechin, epicatechin, gallic acid and epigallocatechin gallate (EGCG). In this case, the phenol species may be conjugated to the polymer or oligomer by forming a conjugate of the polymer or oligomer with an acetal compound (e.g. a dialkyl acetal compound) to form an acetal-functional polymer or oligomer, and coupling the acetal-functional polymer or oligomer with the phenol species. For example, if the polymer is HA and the phenol species is EGCG, then EGCG may be coupled with a HA-acetal (e.g. HA-dialkylacetal) conjugate. This may be accomplished by conversion of the acetal functional group of the acetal-functional polymer or oligomer with an acid to generate an aldehyde functional group. The HA-dialkyl acetal may be formed by reaction of HA with an aminofunctional acetal (e.g. dialkylacetal), such as aminoacetaldehyde diethylacetal. This reaction may be conducted in aqueous solution under acidic conditions, commonly mildly acidic conditions (e.g. pH between about 4 and about 6), optionally in the presence of a condensation reagent such as N-hydroxysuccinimide and/or a carbodiimide. The reaction may be conducted at room temperature or at an elevated temperature, and may take from about 1 and about 24 hours, depending on the reagents, concentrations and temperature. The resulting HA-acetal conjugate may be purified by any of the well known methods, for example dialysis. The HA-acetal conjugate may then be hydrolysed using acid. It may for example be dissolved in water and the resulting solution hydrolysed by adjusting to pH below about 2 (e.g. about 1). This may be accomplished using a strong acid, e.g. a mineral acid such as hydrochloric acid, sulfuric acid or some other convenient acid. Addition of the phenol species (erg. EGCG), optionally in solution (conveniently in a water miscible organic solvent such as DMSO, DMF etc.), to the resulting solution may result in production of the desired HA-phenol species conjugate. The latter reaction may be conducted at room temperature, or at some convenient elevated temperature that does not cause deterioration of the reagents or product. The reaction may be conducted under an intert atmosphere e.g. nitrogen, argon, carbon dioxide. It may take between about 1 and 48 hours, depending on the reagents, concentrations and temperature.

The structure of the binder may be backbone-linker-phenol group, where the backbone is derived from the polymeric species, and the phenol group is derived from the phenolic species. The binder may be made by coupling the linker to the polymeric species to form a backbone-linker combination and then coupling the phenol group to the backbone-linker combination, or it may be made by coupling the phenol group to the linker to provide a linker-phenol group combination (or the linker-phenol group combination may be provided from some other source, e.g. it may be available commercially, for example as tyramine) and coupling the linker-phenol group combination with the polymeric species. For example in the case described above, the aminofunctional acetal or the corresponding aminofunctional aldehyde, may be coupled to EGCG to form an aminofunctional EGCG derivative, and the aminofunctional EGCG derivative may then be coupled to NA to form the HA-EGCG conjugate. The reaction conditions for coupling the aminofunctional EGCG derivative to HA may be similar to those used for coupling the aminofunctional acetal to HA as described above. The reaction conditions for coupling the aminofunctional acetal or aldehyde to EGCG may be similar to those used for coupling HA-dialkyl acetal to EGCG as described above. On curing the cement of the present invention, the backbone-linker-phenol group strut may be converted to a backbone-linker-crosslinked phenol group structure. A partial structure of the backbone-linker-crosslinked phenol group is shown in FIG. 5, however the cured binder of the present invention comprises filler particles distributed within the hydrogel structure shown in FIG. 5.

The binder may for example comprise a polysaccharide having phenolic groups attached thereto, optionally via a linker group (L, as described above), whereby the phenolic groups are capable of crossinking the polysaccharide by an oxidative coupling. The binder may comprise a hyaluronic acid-tyramine (HA-Tyr) conjugate. Other suitable conjugates may be used, for example conjugates with tyramine, catechin, epicatechin, gallic acid or epigallocatechin gallate (EGCG), or mixtures of any two or more thereof. These may be conjugates with hyaluronic acid, or with some other polymer or oligomer.

Alternatively a separate crosslinking species may be mixed with the polymeric species such that the crosslinking species can crosslink the polymer on exposure to a catalyst without evolution of substantial heat. The crosslinking way occur through carbon atoms on an phenol group of the crosslinking species (e.g. through a carbon atom bearing a hydrogen atom before said crosslinking) and/or through an oxygen atom attached to a phenol group of the crosslinking species. A representative crosslinked structure that could be formed by the crosslinking is shown in FIG. 5.

The filler may comprise an inorganic filler, e.g. a mineral filler. It may be a reinforcing filler. It may be non-toxic, and may be biocompatible. It may be non-irritant to a patient treated with the bone cement. It may be for example silica, alumina, zirconia, talc, mica, an apatite or a mixture of any two or more of these. Other suitable fillers are well known to those skilled in the art. Examples of suitable apatite fillers include hydroxyapatite, carbonated apatite and mixtures thereof. The filler may be capable of reacting with the curable binder, or may be incapable of reacting therewith. The filler may have a mean particle size of between about 1 and about 500 microns, provided that the cement (having the filler particles therein) is capable of being injected through a syringe needle. The syringe needle may be between about 18 and 30 gauge. The mean particle size of the filler may be between about 1 and 200 microns, or between about 1 and 100, 1 and 50, 1 and 20, 1 and 10, 1 and 5, 10 and 200, 50 and 200, 100 and 200, 10 and 100, 10 and 50, 200 and 500, 300 and 500, 200 and 300, 100 and 300, 50 and 300 or 50 and 100 microns, for example about 1, 2, 3, 4, 5, 10, 15, 20, 25, 30, 35, 40, 45, 50, 60, 70, 80, 90, 100, 110, 120, 130, 140, 150, 160, 170, 180, 190, 200, 250, 300, 350, 400, 450 or 500 microns. The filler may have a narrow or broad particle size distribution. The filler may have a maximum particle size that is smaller than the internal diameter of the syringe needle (optionally less than 50% or the internal diameter to the syringe needle).

The curing reaction of the cement (i.e. of the curable binder) occurs without substantial evolution of heat. In the context of this specification this is taken to mean that the heat evolved when the cement is cured in the body of a patient may be insufficient to cause damage to surrounding tissue or to components of the curable cement (e.g. proteins that may be incorporated therein). The curing reaction may evolve sufficiently little heat when the cement is cured in the body of the patient (i.e. when it is cured at the body temperature of the patient) that the temperature of the curable cement during the curing reaction does not increase by more than about 5 Celsius degrees, or does not increase by more than about 4, 3, 2, 1 or 0.5 Celsius degrees. The curing reaction may occur at the body temperature of a patient into which it is injected. This temperature will depend on the nature of the patient. It may be between about 35 and about 45° C., or between about 35 and 40, 40 and 45, 37 and 43 or 36 and 39° C., e.g. at about 35, 36, 37, 38, 39, 40, 41, 42, 43, 44 or 45° C. At the curing temperature, the curable cement (when exposed to the curing agent to form the catalysed curable cement) may become solid in between about 10 seconds and about 30 minutes, or 10 seconds and 15 minutes, 10 seconds and 5 minutes, 10 seconds and 2 minutes, 10 seconds and 1 minute, 10 and 30 seconds, 10 and 20 seconds, 30 seconds and 30 minutes, 1 and 30 minutes, 5 and 30 minutes, 10 and 30 minutes, 15 and 30 minutes, 20 seconds and 5 minutes, 20 seconds and 1 minute, 1 and 10 minutes, 1 and 5 minutes or 30 seconds and 2 minutes, for example in about 10, 15, 20, 25, 30, 35, 40, 45, 50 or 55 seconds or about 1, 1.5, 2, 2.5, 3, 3.5, 4, 4, 5, 5, 6, 7, 8, 9, 10, 15, 20, 25 or 30 minutes.

The bone cement may be used for repair of a bone of a patient. The patient may be a vertebrate, e.g. a mammal, a bird, a fish or a reptile. It may be a human or non-human mammal. It may be for example a human, dog, cat, horse, cow, pig, elephant, llama, goat sheep or some other type of mammal.

Curing of the curable binder, and of the curable bone cement, may be promoted by a curing agent. The curing agent may comprise an oxidant. The oxidant may be a mild oxidant so that curing of the cement may be accomplished without substantial evolution of heat. The curing agent may be a reagent for promoting (e.g. catalysing) the oxidative coupling of phenolic groups. The curing agent may comprise an enzyme, e.g. a peroxidase. It may comprise a peroxide. It may comprise a combination of a peroxide and an enzyme e.g. a peroxidase such as horse radish peroxidase (HRP). For example, the curing agent may comprise hydrogen peroxide and horse radish peroxidase.

The bone cement may additionally comprise one or more further component such as collagen, a silicate, a protein (e.g. growth factor) and platelets. The further components may serve to reinforce the cured bone cement, or may serve to promote healing of the bone into which the curable bone cement is injected or of surrounding tissue, or may serve to minimise damage or irritation to surrounding tissue or may serve some other purpose. The further component may be provided in a polymer-inorganic composite drug/protein/growth factor delivery particles in order to deliver healing agents. It may comprise controlled release delivery particles for delivering the healing agents to sites near or adjacent to the region where the cement is injected.

The bone cement (curable or catalysed) may be injectable. It may be in the form of a paste, or a slurry or some other viscous preparation. It may show rheology such that it is injectable using a syringe (e.g. between about 18 and 30 gauge), i.e. at relatively high shear it may be relatively non-viscous (mobile). It may show rheology such that, once injected into a bone, it will not readily flow out of place, i.e. at low shear it may be relatively viscous. It may display a yield stress, such that at shear stresses below the yield stress it does not flow.

The curable bone cement may be made by combining a solution of the curable binder with the filler, and optionally with one or more further component such as collagen, a silicate, a protein (e.g. growth factor) and platelets. The solution may be an aqueous solution. It may comprise additional components for example buffer materials. The solution may be prepared by dissolving the curable binder in a solvent, or may be prepared by combining a solution of a polysaccharide with a reagent, wherein the reagent comprises a crosslinking moiety, such that the polysaccharide reacts with the reagent to form the curable binder. The curable binder should have sufficient crosslinking moieties coupled thereto, or should have sufficient crosslinking species mixed therewith, that the cable cement, once cured to a solid cement, has an acceptable strength and/or hardness. The solid cement may have a wet compressive stiffness of at least about 0.5 MPa, or at least about 1, 2, 5, 10, 50, 100, 200, 300, 400, 500, 600, 700, 800, 900 or 1000 MPa. The wet compressive stiffness may be between about 0.5 MPa and 1 GPa, or between about 1 MPa and 1 MPa, 10 MPa and 1 GPa, 100 MPa and 1 GPa, 500 MPa and 1 GPa, 0.5 and 500 MPa, 0.5 and 100 MPa, 0.5 and 10 MPa, 0.5 and 20 MPa, 0.5 and 10 MPa, 0.5 and 5 MPa, 0.5 and 1 MPa, 1 and 500 MPa, 10 and 500 MPa, 100 and 500 MPa, 10 and 100 MPa or 10 and 50 MPa, and may have a wet compressive stiffness of about 0.5, 1, 1.5, 2, 2.5, 3, 3.5, 4, 4.5, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800 or 900 MPa or about 1 GPa. The crosslink density of the solid cement may be between about 1 and about 50 crosslinks per 100 monomer units of the polymeric species or between about 1 and 25, 1 and 10, 1 and 5, 5 and 50, 10 and 50, 25 and 50, 5 and 25 or 5 and 10 crosslinks per 100 monomer units, e.g. about 1, 2, 3, 4, 5, 10, 15, 20, 25, 30, 35, 40, 45 or 50 crosslinks per 100 monomer units. Thus for example if the curable binder comprises a HA-Tyr conjugate, the molar ratio of HA to Tyr (i.e. to sugar units of the HA) in making the conjugate may be between about 100:1 and 100:50 (based on the sugar units of HA). The solution of the curable binder may be between about 1 and about 10% w/v, or between about 1 and 5, 1 and 2, 2 and 10, and 10, 1 and 3, 2 and 4 or 2 and 3%, for example about 1, 1.5, 2, 2.5, 3, 3.5, 4, 4.5 or 5%. The solution may be combined with the filler in a ratio of between about 1:5 and about 5:1, or between about 1:5 and 1:1, 1:1 and 5:1, 1:4 and 4:1, 1:3 and 3:1, 1:2 and 2:1 or 1:1.5 and 1.5:1, for example about 1:5, 1:4.5, 1:4, 1:3.5, 1:3, 1:2.5, 1:2, 1:1.5, 1:1, 1.5:1, 2:1, 2.5:1, 3:1, 3.5:1, 4:1, 4.5:1 or 5:1 on a w/w basis. The solution of the curable binder and the filler may be combined e.g. mixed, blended, homogenised, vortexed etc. to form the curable bone cement. If further components are included in the cement, they may be added after combining with the filler or before, or at the same time. It will be readily understood that the order of addition at this stage is not critical, and any convenient order may be employed. The further components may be added neat or in solution (e.g. aqueous solution), and if more than one further components are used, they may be added together or separately. For example the further component may be added to the combined curable binder and filler, or the curable binder may be combined with the combined filler and further component (optionally in solution). The ratio of filler to further component may depend on the nature of the filler and of the further component. The ratio may be for example between about 1:2 and about 100:1 on a w/w basis, or between about 1:2 and 50:1, 1:2 and 20:1, 1:2 and 10:1, 1:2 and 5:1, 1:2 and 2:1, 1:2 and 1:1, 1:1 and 100:1, 10:1 and 100:1, 50:1 and 100:1, 1:1 and 50:1, 1:1 and 20:1, 1:1 and 10:1, 1:1 and 5:1, 1:1 and 2:1, 5:1 and 50:1, 5:1 and 20:1 or 5:1 and 0.10:1, for example about 1:2, 1:1.5, 1:1, 1.5:1, 2:1, 2.5:1, 3:1, 4:1, 5:1, 6:1, 7:1, 8:1, 9:1, 10:1, 15:1, 20:1, 25:1, 30:1, 40:1,50:1, 60:1, 70:1, 80:1, 90; 1 or 100:1 or some other ratio.

In order to form a catalysed bone cement, the curable bone cement is exposed to the curing agent. The curing agent may be combined with, e.g. mixed with, stirred with, shaken with, blended with, sonicated with or otherwise combined with the curable cement. The curing agent may be added in sufficient quantity that the bone cement cures at the temperature of use in the desired time. Temperatures and times for curing/setting have been described elsewhere in this specification. This quantity will depend on the nature of the curable cement and of the curing agent. As an example, if the curable cement comprises an HA-Tyr conjugate and the curing agent comprises HRP and hydrogen peroxide, the HRP may be added to the HA-Tyr at between about 0.01 and about 0.05 Units/mg (or between about 0.01 and 0.03, 0.01 and 0.02, 0.02 and 0.05, 0.03 and 0.05, 0.02 and 0.04 or 0.02 and 0.03, e.g. about 0.01, 0.015, 0.02, 0.025, 0.03, 0.035, 0.04, 0.045 or 0.05 Units/mg) and the hydrogen peroxide may be added at about 0.5 and 5 nmol/mg, or between about 0.5 and 2, 0.5 and 1, 1 and 5, 2 and 5, 1 and 3 or 0.8 and 1.2 nmol/mg, e.g. 0.5, 0.6, 0.7, 0.8, 0.9, 1, 1.1, 1.2, 1.3, 1.4, 1.5, 2, 2.5, 3, 3.5, 4, 4.5 or 5 nmol/mg. The HRP and the hydrogen peroxide may each be added in solution e.g., aqueous solution. They may be added together or separately. The concentration of HRP in the solution thereof may be between about 10 and about 100 U/ml (or between about 10 and 50, 10 and 20, 20 and 100, 50 and 100, 20 and 80, 15 and 30, 20 and 30 or 22 and 28 U/ml, e.g. about 10, 15, 20, 25, 30, 35, 40, 45, 50, 55, 60, 65, 70, 75, 80, 85, 90, 95 or 100 U/ml). The concentration of hydrogen peroxide in the solution thereof may be between about 1 and 10 mM, or between about 1 and 5, 1 and 2, 2 and 10, 5 and 10, 2 and 8, 3 and 7 or 4 and 6 mM, for example about 1,2,3,4,5,6,7,8,9 or 10 mM.

The curable bone cement may therefore be combined with the curing agent to form a catalysed bone cement. The cement may then be applied to the bone to be repaired, e.g. it may be injected into the bone or onto, the bone or both. This should be accomplished before the curing reaction has proceeded to the point where the cement is no longer injectable. This will depend on the curing time, which is described elsewhere in this specification. It will be understood that commonly the curing reaction will be accelerated at elevated temperatures. Thus the catalysed bone cement may be prepared at relatively low temperatures (e.g. between about 10 and about 25° C. or 10 and 20, 10 and 15, 15 and 25, 20 and 25 or 15 and 20° C., for example about 10, 15, 20, 25° C. or ambient temperature), at which the curing rate is relatively slow, and may then be injected into a patient with a body temperature between about 35 and 45° C., as described earlier, at which the curing rate may be more rapid.

In one form, the present invention provides an injectable bone cement material comprising of hyaluronic acid-tyramine (HA-Tyr) conjugates and apatites. This injectable paste is capable of setting quickly via the formation of crosslinked network of HA in the presence of horseradish peroxidase (HRP) and hydrogen peroxide. The system shows no, or low, heat release during the formation of bone cements and no, or negligible, or acceptably low, tissue damage because the crosslinking reaction occurs by enzymatic oxidative reaction of the moiety in the HA-Tyr conjugates under mild conditions. This novel injectable HA-apatite-based bone cement is particularly well-suited for the healing of osteochondral defects as it contains mainly HA, collagen and apatites, all of which are native to the bone and joint regions.

HA is a glycosaminoglycan comprised of linear, unbranched, polyanionic disaccharide units. The disaccharide units consist of glucuronic acid N-acetyl glucosamine units joined alternately by beta-1,3 and beta-1,4 glycoside bonds. Tyramine is 4-(2-aminoethyl)phenol.

The curable bone cement of the invention may comprise added collagen, silicates, and/or proteins such as growth factors and platelets. The cement forms an injectable paste (i.e. a catalysed bone cement) when mixed with a solution containing HRP and hydrogen peroxide. It sets within a short time to form a solid material by the crosslinking of the HA-Tyr conjugates. The main advantage of the bone cement of the present invention over traditional injectable bone cements is that the setting process does not release heat, which would damage the surrounding tissues. Evolved heat may also damage components of the cement, for example included growth factor.

The cement of the present invention provides many benefits: (i) it does not require surgical implantation, (ii) it prevents tissue damage, (iii) it suffers less loss in biological activity for growth factors, and (iv) it provides for improved biocompatibility.

From the standpoint that the tissue surrounding the bone is mainly composed of HA and collagen, a bone cement according to the present invention, made using HA-Tyr conjugate with collagen, possesses the advantage that it enables the crystallization of apatite in the HA-collagen matrix without tissue damage. While many bone scaffolds containing HA and collagen have been reported, this bone cement is more versatile as it is possible, using his cement, to regenerate the bone tissue by a simple injection, without damaging surrounding tissue. The bone cement is also particularly well-suited to the healing of osteochondral defects as it contains mainly HA, collagen and apatites, all of which are native to the bone and joint regions. The bone cement may be especially suitable for use at the bone-joint interface as it primarily contains HA and apatites, which are the major constituents of cartilage and bone, respectively. It can be used as a graded composite structure for healing defects at this location.

Animal studies on mice have indicated that a bone cement according to the present invention was non-toxic and biocompatible, and set readily in vivo. In addition, the material also appeared to be osteoinductive as positive alkaline phosphatase staining results were obtained on the extracted samples 5 weeks post-injection.

### EXAMPLES

### Materials and Methods

Hydroxyapatite (HAP) and carbonated apatite (CAP) were synthesized from calcium nitrate, ammonium phosphate and ammonium carbonate by base precipitation. Collagen was extracted from rats, and dissolved in 0.05 M phosphoric acid at a concentration of 40 mg/ml. Four different formulations of injectable pastes were examined:

1. HA-Tyr solution only (control)

2. HA-Tyr solution and apatite powders

3. HA-Tyr solution and apatite powders, and collagen solution

4. HA-Tyr solution, and pro-mixed collagen-apatite solution HA-apatite-based bone cements, both with and without collagen, set in mice by injection of the paste mixture of HA-Tyr, apatite, HRP and hydrogen peroxide. For the sample without collagen, HA-Tyr (25 mg) was dissolved in 1 ml of PBS (phosphate buffer solution). To this solution, 600 mg of apatite powder was added, followed by vortexing thoroughly. Freshly prepared 25 μl of HRP (25 U/ml) and 5 id of hydrogen peroxide 0.14 mol/L) solutions were added to the paste of HA-Tyr as curing agent for the enzymatic oxidative coupling reaction. The paste was then injected subcutaneously through an 18-gauge needle into the Swiss albino mice where it set into a solid cement within 30 seconds from the time of addition of HRP and hydrogen peroxide. For the sample with collagen, we prepared two different paste solutions: (i) the paste solution of HA-Tyr and apatite containing 0.5 ml of collagen, and (ii) HA-Tyr solution containing 1 ml of premixed solution of collagen and apatite.

5 weeks post-injection, the mice were sacrificed and the injected cement was removed for cryosectioning and histological analysis. The slides were immunostained using hematoxylin and eosin (H and E), alkaline phosphatase and nuclear fast red (ALP and NFR), and Von Kossa and nuclear fast red (VK and NFR) solutions.

### Results and Discussion

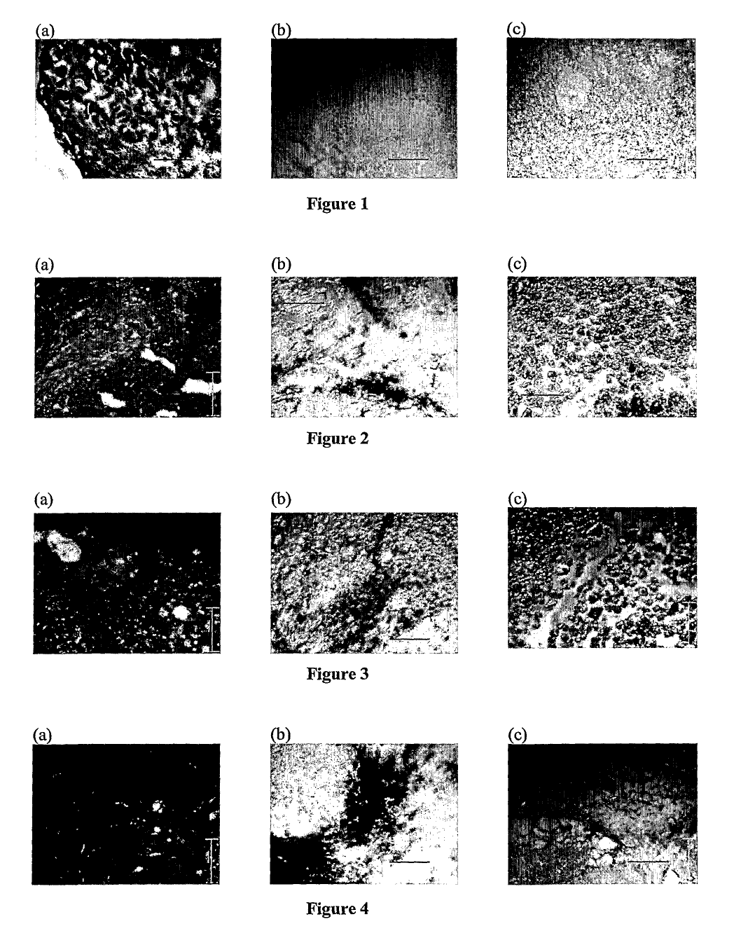

After 5 weeks post-injection, the following results were obtained. H and E staining showed that there was healthy cell proliferation, blood supply and tissue ingrown with no necrosis for all samples (FIGS. 1(a), 2(a), 3(a) and 4(a)). (H and B is Hematoxylin and Eosin stain for histological tissue sections. Cell nuclei will be stained blue, with some metachromasia. Cell cytoplasm will be stained various shades of pink, identifying different tissue components. ALP is Alkaline Phosphatase Chromogen stain for histological sections (also known as BCIP/NBT; BCIP: 5-bromo-4-chloro-3-indolyl phosphate, NBT: p-nitroblue tetrazolium chloride). Areas with alkaline phosphatase activity will be stained a deep purple. Alkaline phosphatases are a group of enzymes found primarily the liver (iso ec ALP-1) and bone (isoenzyme ALP-2). NFR is Nuclear Fast Red stain, a counterstain for histological sections. Cell nuclei will be stained red and cell cytoplasm will be stained pink. VK is Von Kossa staining of histological sections for calcium. This technique is for demonstrating deposits of calcium or calcium salt, so it is not specific for the calcium ion itself. In this method, tissue sections are treated with a silver nitrate solution and the silver is deposited by replacing the calcium reduced by the strong light, and results in a black or brown-black stain in areas with calcium salts.) Compared to the control (FIG. 1(b)), the incorporation of apatites into the material formulation resulted in positive ALP staining, where areas of osteoblast activity were stained dark purple (FIGS. 2(b), 3(b) and 4(b)). Positive VK staining (dark brown) was also observed in the samples containing apatites (FIGS. 2(c), 3(c) and 4(c), which could be due to the calcium present in the apatites or released through osteoblast activity. This indicated that our materials were non-toxic and biocompatible. In addition, the apatite-containing formulations also appeared to be osteoinductive since ALP activity was observed after injection into an ectopic region.

### CONCLUSIONS

The inventors have synthesized bone cement materials that are injectable and fast-setting in vivo with no heat release or surrounding tissue damage. A simple and non-toxic injectable in situ bone cement system was achieved using an enzymatic oxidative coupling reaction. The biocompatibility and convenience of application of this injectable bone cement system would be highly advantageous to the healing and regeneration of bone defects.

Preliminary in vivo studies confirmed that the HA-apatite-based materials were non-toxic and biocompatible, and likely to be osteoinductive. These bone cements contain primarily hyaluronic acid and apatites, both of which are naturally abundant in the bone-joint area. These characteristics would make the materials particularly well-suited for the healing of defects in the osteochondral region, and for use in spinal fusion, bone and joint defects, osteoporotic fractures, maxillofacial and revision surgery, and vertebroplasty.

### Synthesis Of Hyaluronic Acid-Aminoacetylaldehyde Diethylacetal Conjugate (1)

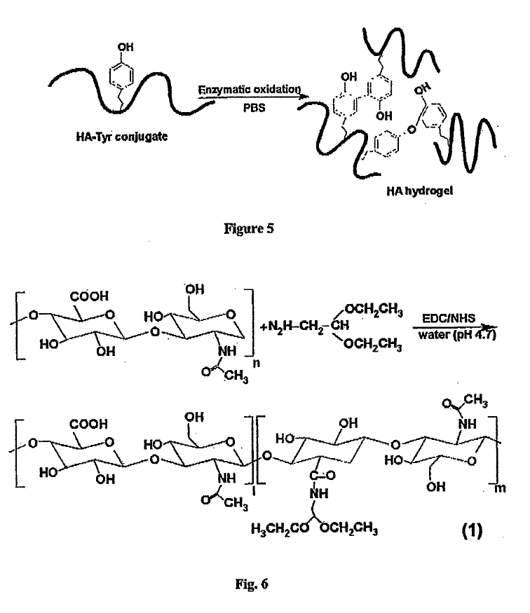

The conjugate (1) was synthesized by following a general protocol, which is shown in FIG. 6. HA (1 g, 2.5 mmol) was dissolved in 100 ml of distilled water. To this solution aminoacetaldehyde diethylacetal (1.2 g, 9 mmol) was added. The pH of the reaction mixture was adjusted to 4.7 by the addition of 0.1 M HCl. N-hydroxysuccinimide (0.34 g, 3.0 mmol) and 1-ethyl-3-[3-dimethylamino)propyl]carbodiimide hydrochloride (EDC) (0.575 g, 3.0 mmol) were added to the solution. After mixing, the pH of the reaction was maintained at 4.7. The solution was kept at room temperature for 24 h under gentle stirring. The mixture was subjected to purification by dialysis (molecular weight cut off=1000).

### Synthesis Of Hyaluronic Acid-Epigallocatechin Gallate (Ha-EGCG) Conjugate

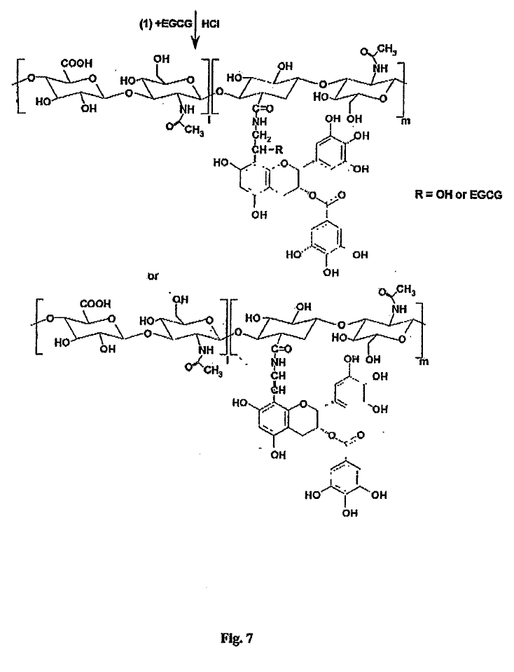

HA-EGCG conjugate was synthesized by the protocol shown in FIG. 7. 1 g of conjugate (1) was dissolved in 60 ml of distilled water. Then the pH of the solution was adjusted to 1 by adding HCl solution. To this solution 5 ml of EGCG solution dissolved in DMSO (0.2 g/ml) was added. The solution was kept at room temperature under nitrogen for 24 h under gentle stirring. The mixture was subjected to purification by dialysis (molecular weight cut off=1000).

1. A curable bone cement comprising a curable polymeric binder and a filler, wherein the cement is capable of curing without substantial evolution of heat on exposure to a curing agent, said binder comprising phenol groups which are capable of reacting in order to cure the cement.

2. The curable bone cement of claim 1 wherein the phenol groups comprise —C₆R′₄OR groups, wherein R and each R′ are independently hydrogen, an alkyl group, an aryl group or an acyl group, and R′ may also be OH, and each R′ is the same as or different to each other R′, provided that at least one R′ ortho to the OR group is hydrogen, and wherein R and R′ are such that one —C₆R′₄OR group is capable of oxidatively coupling with another —C₆R′₄OR group.

3. The curable bone cement of claim 2 wherein at least some of the —C₆R′₄OR groups are —C₆H₄OH groups.

4. The curable bone cement of claim 1 wherein the curable polymeric binder comprises a conjugate of a polysaccharide, a polyamine or a polypeptide with a compound selected from the group consisting of tyramine, catechin, epicatechin, gallic acid and epigallocatechin gallate (EGCG), or with a mixture of any two or more thereof.

5. The curable bone cement of claim 4 wherein the polysaccharide is hyaluronic acid.

6. The curable bone cement of claim 1 wherein the curing agent comprises an oxidant.

7. The curable bone cement of claim 1 wherein the curing agent comprises an enzyme.

8. The curable bone cement of claim 7 wherein the enzyme is a peroxidase enzyme.

9. The curable bone cement of claim 7 wherein the curing agent additionally comprises a peroxide.

10. The curable bone cement of claim 1 wherein the curing agent comprises hydrogen peroxide and horse radish peroxidase.

11. The curable bone cement of claim 1 wherein the cement is capable of curing to a solid in between about 10 seconds and about 30 minutes without substantial evolution of heat on exposure to the curing agent at the body temperature of a patient in which the cement is cured.

12. The curable bone cement of claim 1 wherein the filler comprises a mineral filler.

13. The curable bone cement of claim 1 wherein the filler comprises an apatite or a mixture of two or more apatites.

14. The curable bone cement of claim 1 wherein the filler comprises a material selected from the group consisting of hydroxyapatite, carbonated apatite, fluoroapatite, a modified apatite, silica, calcium phosphate, alumina, zirconia, talc, mica and mixtures thereof.

15. The curable bone cement of claim 1 additionally comprising at least one further component selected from the group consisting of collagen, a silicate, a protein and platelets.

16. The bone cement of claim 15 wherein the protein is a growth factor.

17. A catalysed bone cement comprising the curable bone cement of claim 1 combined with the curing agent.

18. The bone cement of claim 17 which is injectable.

19. The bone cement of claim 17 which is in the form of a paste.

20. A process for making a curable bone cement comprising combining a solution of a curable polymeric binder and a filler, said binder comprising phenol groups which are capable of reacting in order to cure the cement, whereby the cement is capable of curing without substantial evolution of heat on exposure to a curing agent at the body temperature of a patient in which the cement is cured.

21. The process of claim 20 wherein the phenol groups comprise —C₆R′₄OR groups, wherein R and each R′ are independently hydrogen, an alkyl group, an aryl group or an acyl group and each R′ is the same as or different to each other R′, provided that at least one R′ is hydrogen, and wherein R and R′ are such that one —C₆R′₄OR group is capable of oxidatively coupling with another —C₆R′₄OR group

22. The process of claim 20 wherein the curable polymeric binder comprises a conjugate of a polysaccharide, a polyamine or a polypeptide with a compound selected from the group consisting of tyramine, catechin, epicatechin, gallic acid and epigallocatechin gallate (EGCG)₇ and mixtures of any two or more thereof.

23. The process of claim 20 wherein the filler comprises an apatite, a mixture of apatites, silica, calcium phosphate, alumina, zirconia, talc, mica or a mixture of two or more of these and the curing agent comprises an enzyme.

24. The process of claim 23 wherein the enzyme is a peroxidase enzyme.

25. The process of claim 23 wherein the curing agent additionally comprises a peroxide.

26. The process of claim 20 comprising adding at least one further component selected from the group consisting of collagen, a silicate, a protein and platelets.

27. A method for curing a curable bone cement, said method comprising:
exposing the curable bone cement to a curing agent to form a catalysed bone cement; and
curing the catalysed bone cement without substantial evolution of heat;
wherein the bone cement comprises a curable polymeric binder and a filler, and wherein the cement is capable of curing without substantial evolution of heat on exposure to the curing agent at the body temperature of a patient in which the cement is cured, said binder comprising phenol groups which are capable of reacting in order to cure the cement.

28. The method of claim 27 wherein the curing agent comprises an enzyme.

29. The method of claim 27 additionally comprising the step of injecting the bone cement into a patient before the step of curing the catalysed bone cement.

30. A method for at least partially repairing a bone in a patient comprising:
combining a curable bone cement with a curing agent to form a catalysed bone cement,
injecting said catalysed bone cement onto and/or into said bone; and
curing the catalysed bone cement on and/or in the bone without substantial evolution of heat;
wherein the bone cement comprises a curable polymeric binder and a filler, and wherein the cement is capable of curing without substantial evolution of heat on exposure to the curing agent at the body temperature of the patient, said binder comprising phenol groups which are capable of reacting in order to cure the cement.
